(19) United States
(12) Patent Application Publication
(10) Pub. No.: US 2026/0224839 A1
(43) Pub. Date: Aug. 6, 2026

(54) STRESS-REDUCING LIGHTING

(71) Applicants: The Regents of the University of California, Oakland, CA (US); Toyota Boshoku America, Inc., Erlanger, KY (US)

(72) Inventors: Michael J. Siminovitch, Woodland, CA (US); Jae Yong Suk, Davis, CA (US); George R. Mangun, Davis, CA (US); Sreenivasan Meyyappan, Davis, CA (US); Camelia E. Hostinar Caudill, Davis, CA (US); Vijayavel K. Ramachandran, Canton, MI (US); Kristi K. Doherty, Davison, MI (US)

(73) Assignees: The Regents of the University of California, Oakland, CA (US); Toyota Boshoku America, Inc., Earlanger, KY (US)

(21) Appl. No.: 19/149,675

(22) PCT Filed: Feb. 12, 2024
(86) PCT No.: PCT/US2024/015420

§ 371 Date: Jul. 21, 2025

Related U.S. Application Data
(60) Provisional application No. 63/484,648, filed on Feb. 13, 2023.

(51) Int. Cl. A61M 21/00; B60Q 3/70; H05B 45/20

(52) U.S. Cl. CPC A61M 21/00; B60Q 3/70; A61M 2021/0044; H05B 45/20

(57) ABSTRACT

Apparatus, systems, and methods are provided for stress reduction and/or other beneficial health effects in a human by controlling one or more properties of lighting observed by the human, such as color (or wavelength) and/or intensity. Area lighting (e.g., for a room, a portion of a room, a vehicle) and/or specific equipment (e.g., medical equipment and devices) may incorporate an apparatus or system provided herein. During operation, a lighting fixture or other source outputs lights in selected regions of the visible light spectrum, such as an amber region (e.g., 570-680 nm), a red region (e.g., beyond 680 nm), and a green region (e.g., 510-540 nm), while eschewing other regions (e.g., below 510 nm). Light emitted from a system or apparatus may account for some, most, or all radiant flux encountered by the human, and the stress-reduction benefit may be verified via biological or biometric examination.

### BACKGROUND

This disclosure relates to the fields of electronics and lighting. More specifically, methods, apparatus and systems are provided for emitting light in a manner (e.g., in terms of wavelength, color spectrum) designed to reduce stress and/or maintain a relatively unstressed condition within humans.

Medical and dental patients, students, commuters, office workers, and other people are subject to varying degrees of stress due to their health, school obligations, traffic, work requirements, etc. Many environments in which people feel stress are lighted to enhance safety or to provide illumination for reading and/or physical tasks, but the lighting may aggravate one's stress. For example, bright lighting may aide performance of physical tasks, but may be so bright as to be glaring and visually uncomfortable.

Background and area illumination have been used almost exclusively to support visual functionality using light wavelengths in the range of 300 to 700 nm. Visual functionality relates to the performance of components of the human visual system, including spectral (color) and contrast discrimination functions, as well as contrast sensitivity (the ability to see contrast). Enhanced spectrum and increased intensity levels within the typical visual spectrum can be used to support visual functions principally for visual performance issues.

Although some professional environments have been designed to improve comfort by implementing soothing interior design, wall/floor/ceiling colors, comfortable furniture, glare-free lighting and/or dimmable lighting, heretofore the lighting color or spectra have not been intelligently adjusted to provide additional benefit, such as to alleviate stress.

### SUMMARY

In some embodiments, methods and apparatus are provided to emit light in one or more color ranges discovered to measurably reduce or mitigate stress in human beings, as well as to maintain a relatively unstressed condition. More specifically, the light spectra implemented in these embodiments may reduce stress markers and cortisol in people exposed to the lighting, and may also help produce positive brain wave patterns. These embodiments may be particularly useful and effective in medical and/or dental environments, where a patient may be receiving treatment or undergoing a stressful examination (e.g., using an MRI or Magnetic Resonance Imaging machine), or may be recovering from such a treatment or examination. They may also be of significant assistance in other settings that may be stressful (e.g., while driving, at work, while studying).

In some embodiments, an area lighting system for a room or other space, or a system comprising one or more individual lighting devices, is configured to produce light within one or more desired spectra, permanently or on a timed basis. For example, lighting in one color region may be provided for one period of time, followed by lighting in a different color region for a second period of time, and so on.

Also, or instead, individual lighting devices or apparatuses may be configured to produce light as described herein. For example, medical devices or equipment (e.g., MRIs, CT (Computerized Tomography) scanners, blood transfusion equipment, pre/post-operative waiting rooms, etc.), lamps, spotlights, and/or other lighting hardware may be augmented with the ability to provide light in one or more color regions. Similarly, a relatively small area within a larger space may be lighted using beneficial light spectra, while some or all the remainder of the larger space may be lighted in some other manner.

Therefore, in different implementations, light produced in a particular color region may account for a different portion or percentage of the total radiant flux from some or all light sources lighting a given space or area. For example, in a medical recovery room, lighting may be configured or adjusted so that no less than 80% of the total flux is in an amber region (e.g., 570-680 nm). Other illustrative conditions or constraints may include (a) no more than 5% of total flux within another region (e.g., 510-540 nm), (b) no more than 10% of total flux at wavelengths above 680 nm, and/or (c) no light flux below 510 nm.

As another example, for an area lighting system for a room, 50 to 100 percent of the total radiant flux emitted by the area's lighting hardware may be concentrated at approximately 590 nm (amber), plus or minus some margin (e.g., 50 nm). For a smaller area, such as when a lighting system provided herein is embedded in a medical device or apparatus, the colored (i.e., non-white) light output may be an even higher percentage of total radiant flux (e.g., up to 100 percent).

Embodiments may be implemented in private and/or public areas, and may provide particular benefit in places such as medical offices and waiting rooms, surgery spaces, recovery rooms, break rooms for medical and professional staff, treatment areas for conditions such as post-traumatic stress disorder, psychiatric illness, bereavement, pediatric conditions, etc. Further, individual apparatus (e.g., lamps, overhead lights) that illuminate a limited area may provide benefit to individuals engaged in stressful tasks. Thus, a student's study area, or at least the portion of the area that he or she sees while studying, may be illuminated with one or more lighting devices that output light spectra described herein. Similarly, an automobile may emit interior light using a custom light spectrum described above, in some or all areas that may be viewed by a driver and/or passenger.

### DESCRIPTION OF THE FIGURES

FIG. 1 illustrates recovery rates of stressed subjects while exposed to different light spectra, according to some embodiments.

FIG. 2 is a flowchart demonstrating a method of applying a spectral formula, in accordance with some embodiments.

FIGS. 3A-B illustrate example lighting apparatus that may be used to emit light according to a specific spectral formula for reducing stress, in accordance with some embodiments.

### DESCRIPTION

The following description is presented to enable any person skilled in the art to make and use the disclosed embodiments, and is provided in the context of one or more practical applications and their requirements. Various modifications to the disclosed embodiments will be readily apparent to those skilled in the art, and the general principles defined herein may be applied to other embodiments and applications without departing from the scope of those that are disclosed. Thus, the present invention or inventions are not intended to be limited to the embodiments shown, but rather are to be accorded the widest scope consistent with the disclosure.

In embodiments disclosed herein, systems, apparatus and methods are provided for reducing stress and/or providing other health benefits through intelligent lighting, especially in terms of the color spectra used to light a room, a portion of a room, a task, a piece of medical equipment, or some other space or item. Different colors or wavelengths of light may be combined in different proportions or ratios while reducing stress and anxiety to enhance well-being, and/or to maintain a (relatively) stress-free condition.

In some implementations, discrete light spectra are applied in medical and related health care environments. These applications promote recovery from stressful and/or invasive examination/testing/medical procedures, including MRI (Magnetic Resonance Imaging), CT (Computerized Tomography) scan, X-ray, colonoscopy, outpatient surgery, blood draws, transfusion, and so on. For example, the tubular housing of an MRI machine may be configured to predominantly (or only) feature light within a particular portion of the spectrum to provide an intimate surrounding of stress-reducing color. The room or space in which the machine is located may feature the same color(s) of light, but possibly with different proportions.

Similarly, however, other spaces can be configured with lighting spectra described herein for the purpose of improving the mental well-being of humans situated in the spaces. For example, the interior of an automobile or other vehicle may be designed so that the driver and/or passengers are subjected to beneficial light spectra and intensities. As another example, students' study areas may be designed to provide the same benefit.

Previously, there has been a clear lack of evidence-based data in terms of biomarkers, biological/chemical tests, or other measurable factors that would support the use of stress-mitigating lighting as a recommended practice. Most past studies are anecdotal or involved human subject testing using preference-based study approaches that were often fraught with experimental challenges and/or cultural biases that limited general applicability of the observations.

However, recent research conducted in association with development of embodiments described herein has demonstrated non-visual implications of light on health and well-being, mainly relating to circadian and hormonal balance. For example, the melanopsin receptor is particularly sensitive to spectral content within the 480 nm region (blue-green), which leads to appropriate suppression of melatonin and circadian entrainment. However, at night, excessive exposure to high light levels, particularly in the blue-green spectral region, can be deleterious to the circadian system by suppressing melatonin. Conversely, low levels of light at night can mitigate circadian disruption. It has also been shown that near-IR (infrared) radiation in the range of 750-1,200 nm can support circadian entrainment.

Embodiments described herein leverage the results of a human subject-controlled study of the use of discrete spectra for stress mitigation or recovery from a stressful event. The principal aim of this research was to identify spectra that would reduce cortisol and achieve enhanced brainwave patterns that indicate stress reduction. In the study, stress conditions were introduced to individual subjects with their brain wave patterns and cortisol levels being monitored before, during, and after (e.g., with EEG (electroencephalography) and saliva tests, respectively), and the subjects were exposed to discrete light spectra after being stressed. The experiment was reproduced for a series of colors.

For the testing, a color isolation chamber was employed with the potential to introduce any color spectra. In this chamber, the lighting system and apparatus provided only indirect light, meaning that subjects saw only reflected light and no naked or bare LEDs, light bulbs, or other intense light sources. The lighting system illuminated the entire chamber so that the chosen light spectra was the only light visible to the subjects.

Each cohort of subjects was stressed using standardized techniques. The subjects were then exposed to specific color spectra and cortisol/brainwave patterns were monitored throughout. The results demonstrated that: (1) amber light spectra introduced discreetly were able to produce conditions that mitigated or reduced the subjects' stress; and (2) examination of front alpha (8-12 Hz) asymmetry between right and left brain hemispheres (using EEG) indicated that light concentrated in the amber region has the most significant and positive impact in terms of reduced stress.

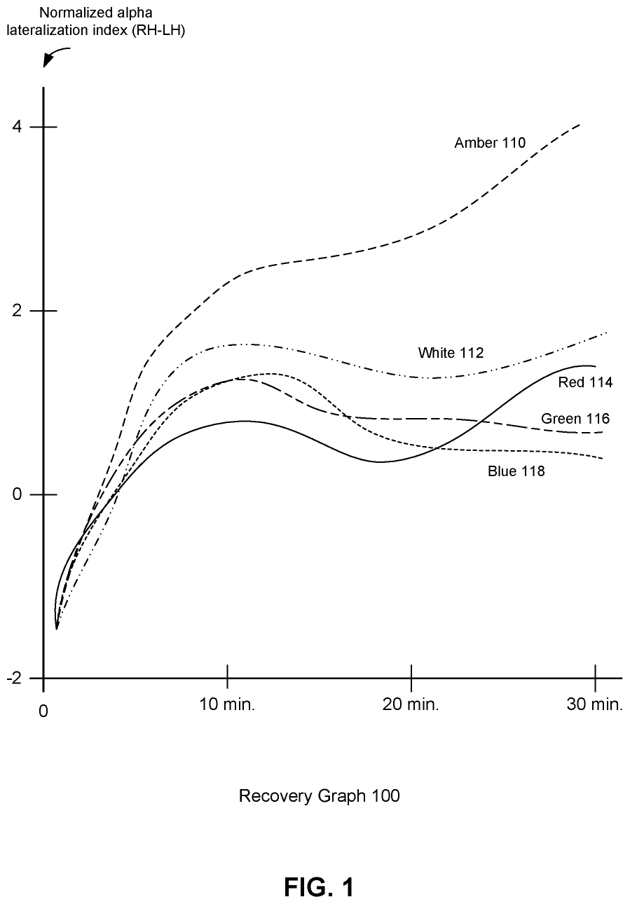

FIG. 1 illustrates recovery rates of stressed subjects of the study described above while exposed to different light spectra. Recovery graph 100 graphs normalized alpha lateralization indices (RH-LH) over time for each of multiple spectra, represented as amber 110, white 112, red 114, green 116, and blue 118. Amber plot 110 corresponds to recovery light in the range of 570-680 nm, white plot 112 corresponds to recovery light in the range of ??, red plot 114 corresponds to recovery light in the range of 440-700 nm, green plot 116 corresponds to recovery light in the range of 500-560 nm, and blue plot light 118 corresponds to recovery light in the range of 440-480 nm. Graph 100 thus demonstrates that applying light within discrete spectra to people recovering from a stressful event had clear impact.

The study was conducted in a closed room, approximately 8 feet wide by 8 feet long by 9 feet high. The walls and ceiling featured a matte white finish with high reflectance (approximately 80% to 85%). Two rows of four-channel color-changing RGBA (red, green, blue, alpha channel (for expressing opacity)) lighting fixtures were mounted on the four walls horizontally at 6.5 feet above the finished floor (AFF) to produce indirect lighting reflected from the ceiling and the upper and lower portions of the walls. A total of sixteen 4 ft long fixtures with 100°×100° beam optics were installed inside the room, but hidden behind fascia that extended 4 inches away from the walls.

This configuration helped avoid potential glare to participants and helped create evenly distributed illumination throughout the entire space. More specifically, the illuminance of the lighting (e.g., when an amber spectrum was emitted) was 50 lux (about 5 foot-candles) at task height (approximately 2.5 ft AFF)-sufficient to be visually comfortable without high contrast and without direct line of sight with the lighting apparatus.

Beyond identifying light spectra that are particularly effective at reducing stress (i.e., amber light in the range of 570 to 680 nm (and especially 580-600 nm)), the study also showed that avoiding or limiting other spectra are advisable. For example, although the effect of amber wavelengths of light is most pronounced when the amber light constitutes 100% of the light flux perceived by a stressed person, it may be decreased to approximately 80-85% of total flux and still supply significant benefits.

However, it was also determined that, when the amber light spectra accounts for less than 100% of the flux, a maximum of 10% of the total flux should be at wavelengths beyond amber (i.e., above 680 nm), a maximum of 5% of the total flux should be in a green wavelength region (e.g., 510-540 nm), and no flux less than 510 nm (which corresponds to cyan blue) should be included.

Within these boundaries, the lighting for an area, a task, a machine, etc., may be adjusted for purposes other than stress mitigation or reduction. For example, the specific composition of light experienced by a person may be designed to affect a person's ability to perform a task (e.g., drive, read, work with a tool).

Allowing up to 15% of light spectra to be outside the amber region may yield a more natural setting that allows a person to better observe colors and patterns and to perform desired tasks. Of course, when the primary or only concern is to improve a person's mental condition during or after a stressful event or process, a narrower spectral distribution (at or near 100% amber) may be desired.

Therefore, lighting apparatus and systems described herein may be programmed to emit light according to these constraints and/or others (e.g., a level of stress detected or observed, a time period during which the lighting apparatus/system can operate). For example, a location or space in which stress-mitigating lighting is installed may include one or more sensors that automatically sample light and determine its composition. When the portion of flux within the amber region falls or threatens to fall below 80%, a lighting system may cease emitting light at other wavelengths or adjust the ratio at which light in different color regions is emitted. Illustratively, daylight may impinge upon the space (e.g., through windows, around window dressings), white light may spill into the space from an adjacent space or through a door, or the illumination of the space may be altered in some other way. By detecting changes in the lighting, the system can automatically make corrections within programmed thresholds and parameters.

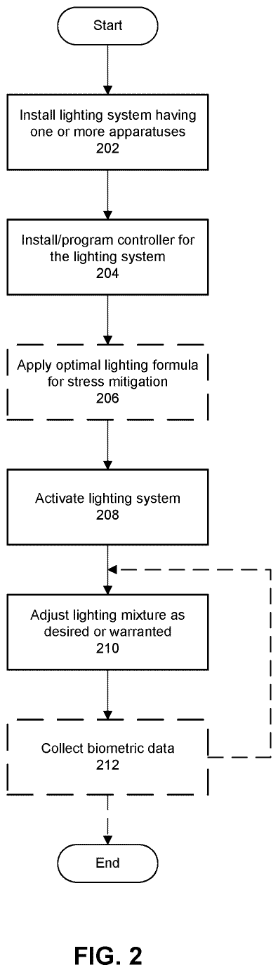

FIG. 2 is a flowchart demonstrating a method of applying a spectral formula to yield stress-mitigation benefits, in accordance with some embodiments. One or more of the illustrated steps may be omitted, repeated, and/or performed in a different order. Accordingly, the specific arrangement of steps shown in FIG. 2 should not be construed in a manner that limits the scope of the embodiments.

In operation 202, a room, a portion of a room, a cubicle, the seating area of a vehicle, a piece of equipment (e.g., medical equipment), or some other space is enhanced with a system comprising one or more apparatuses for emitting light in one or more color (or wavelength) ranges other than (or in addition to) white. For example, one or more lighting devices described further below (e.g., LEDs, lamps, lighting tubes, spotlights) may be installed to support the illustrated method. The apparatuses may be integral to the space or equipment (i.e., built into it when constructed) or may be added on after the space or equipment is constructed.

In operation 204, one or more controllers for controlling operation of the lighting apparatus are programmed or otherwise configured. Although a controller may be included in a lighting apparatus in some implementations, in other implementations the system includes a hardware or software controller (e.g., a DMX controller) that is external to the lighting apparatuses and that may control any or all of the individual apparatuses.

In the illustrated embodiments, programming of the controller may include configuring it with one or more preset options for the light to be emitted from the lighting apparatuses, especially in terms of color/wavelength, but also possibly in terms of intensity, duration of operation, automatic transition from one lighting mixture to another, and so on. A lighting mixture or ratio, as used herein, refers to the mixture or ratio of flux from different portions of the light spectrum that are combined to yield the light output by the lighting system. Thus, a first mixture may be composed solely of amber-colored light. A second mixture may be composed of (approximately) 85% amber light, 5% green light, and 10% red light. Other mixtures may combine any colors with any desired ratios. A controller may also (or instead) be manually operated to change the mixture as desired during operation.

In optional operation 206, the lighting system and/or one or more individual lighting apparatuses are constrained (e.g., via programming of a controller) by a lighting formula that ensures that emitted light is configured to always yield stress mitigation benefits described above. More specifically, when so constrained, during operation of the system or apparatuses, the total emitted light flux will always contain between 80% and 100% of flux that is amber in color (e.g., 570-680 nm). Further, no light below 510 nm will be emitted, no more than 5% of the total light flux will be in a green region (e.g., 510-550 nm), and no more than 10% of the light flux will be at wavelengths beyond 680 nm.

In operation 208, the lighting system is activated manually or automatically.

For example, when part of a piece of medical equipment (e.g., an MRI machine), the lighting system may activate when the equipment is powered on or when a patient is located in or near the equipment. When part of a vehicle, the lighting system may activate when the vehicle is turned on, when it is occupied, or when it begins moving. When part of a room or space, the lighting system may be activated when occupied by one or more humans, when a desire for stress-mitigation is expressed, or on a timed basis (e.g., to operate during certain hours of a day). In some implementations, a stress-reducing lighting mixture is activated when a person activates the controller (e.g., by activating a switch).

In operation 210, the light mixture output by the lighting system may vary over time, automatically and/or manually, subject to any applicable constraints such as those discussed above. For example, when the system illuminates a medical recovery room, the system may initially emit solely (or almost solely) amber light, to provide the maximum stress-reduction benefit. After some period of time (e.g., minutes, hours), the mixture may change to include some other color(s) of light, possibly to facilitate a task (e.g., reading), to display a color that a patient finds soothing or comforting, or for some other reason.

Similarly, a lighting system in another space (e.g., an automobile, a study area) may commence operation with light having less flux in an amber region of the spectrum and more flux of some other discrete color, or white, but then transition over time to increase the proportion of amber light, perhaps as more stress is detected or a stressful event is encountered.

Further, programmed (and/or manual) constraints may be always active or only active at specified times. For example, a room may be illuminated naturally with daylight or white light during a portion of the day, such as in the morning. Later, the constraints may be activated (in conjunction with blinds or other window coverings for blocking daylight, if necessary) to provide the resultant stress reduction or alleviation. Operation 210 may continue indefinitely, or a lighting system may be turned off when not needed (e.g., overnight, between patients).

In optional operation 212, biometric data may be collected from one or more people viewing light from the lighting system. In different implementations, different collection methods may be employed, such as a saliva test, observing pupil size, determining a pulse rate, recognizing a facial expression, or measuring a brainwave pattern via EEG. The data may indicate that the lighting mixture should change to increase or decrease the stress reduction effect (e.g., by increasing or decreasing the proportion of amber light), or may indicate that the current mixture is providing the desired benefit and need not be changed. Thus, after operation 212, the method may return to operation 210 or may end.

A lighting system provided herein may include any number of lighting apparatus capable of emitting light in all desired colors, which may or may not include white light. For example, in the study described above, multiple wall fixtures were employed around the circumference of a closed space (a room). Each fixture comprised four LEDs or LED chips that produced, respectively, red, green, blue, and amber light. A DMX (digital multiplex) controller controlled which LEDs were active at a given time and their relative intensities when a color other than red, green, blue, and amber was desired.

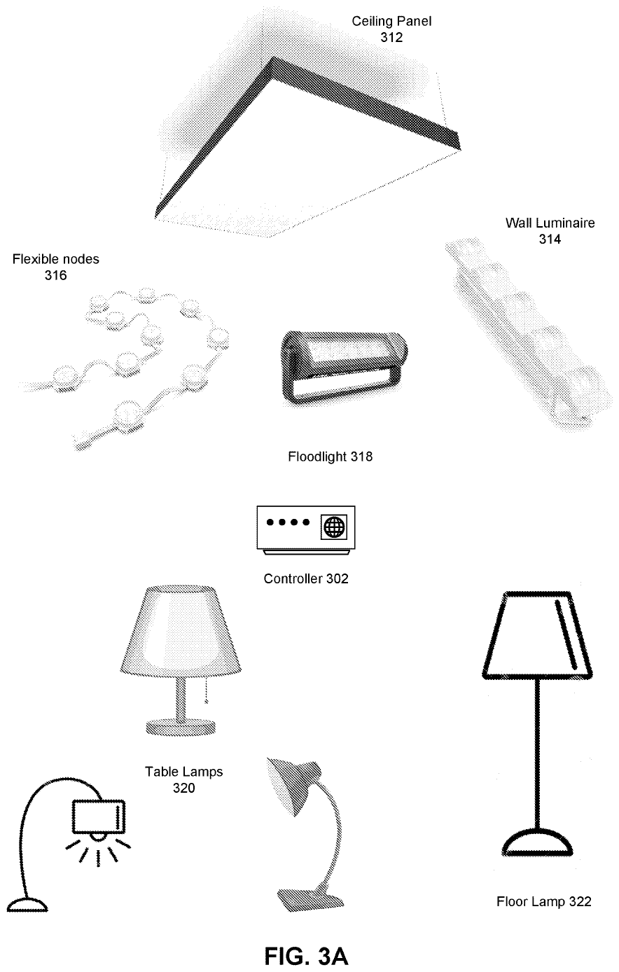

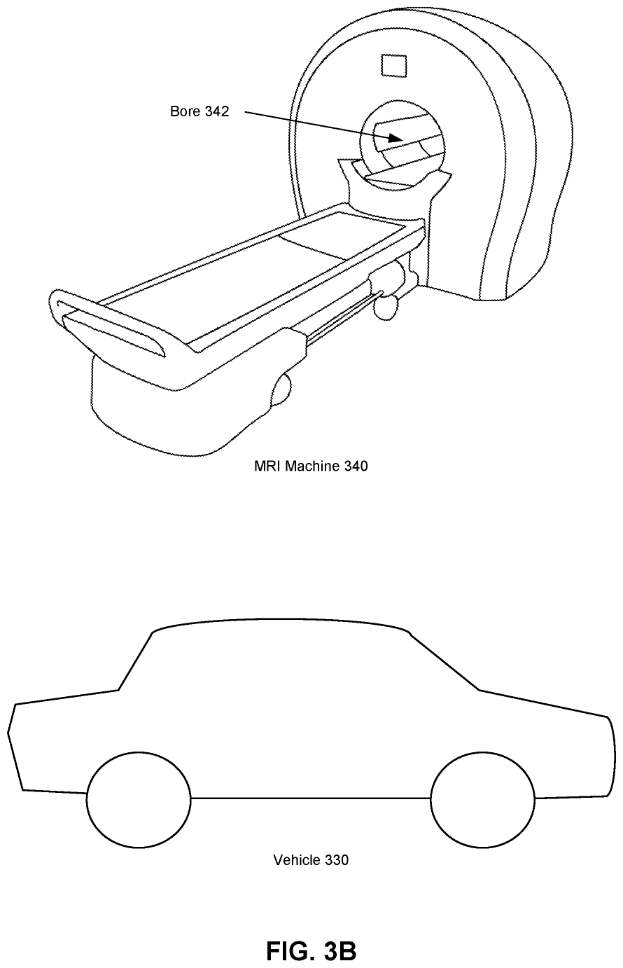

FIGS. 3A-B illustrate lighting apparatus for providing the stress-reduction benefits provided with some embodiments. The apparatus shown in FIG. 3A may be controlled (e.g., in terms of the color and intensity of light they emit) by controller 302; apparatus in FIG. 3B may be controlled by controllers internal to the apparatus or by an external controller such as controller 302. Controller 302 may communicate with individual lighting apparatus via wired and/or wireless technologies and protocols.

One or more ceiling panels 312 can light an area with diffused and colored light and may be suspended from a ceiling or be flush with the surface of a ceiling. Wall luminaires 314 may provide direct light or, as in the study described above, provide indirect light when installed behind fascia or other partial cover.

Flexible nodes 316 provide for irregular placement of lights that provide stress-reducing colored light, and floodlight 318 allows illumination of a relatively large area, either directly or indirectly. Table lamps 320 and floor lamp or torchiere 322 enable emission of stress-reducing light in relatively small areas for local benefit to someone performing a task, for example.

Vehicle 330 (e.g., an automobile) may include lighting apparatus or devices for the benefit of a vehicle operator and/or passengers. Lighting within vehicle 330 may cooperate with a window tinting technology and/or operate primarily when the light emitted by the internal devices is not dominated by external light (e.g., during daytime). In some implementations, stress-reducing lighting in vehicle may be 330 located in the roof to provide diffuse lighting throughout much or all the passenger compartment. In other implementations, the lighting may be emitted from door panels, underneath the dashboard, underneath or behind seating, in the flooring, etc. To avoid distracting or blinding an operator of the vehicle, some or all light sources are obscured or shielded to provide mainly or only indirect light.

In MRI machine 340, stress-reducing lighting is built into (or added to) the bore in which a patient reposes. For example, one or more light sources may be located within the bore and/or may be situated outside the bore and shine into the bore. Different colors and/or intensities of light may be employed outside the bore. For example, within the bore, all or virtually all the light flux perceived by the patient may be in a known color region such as amber. Outside the bore, the flux mixture may employ less than 100% amber, perhaps to aid a technician or medical professional tending to the patient.

Thus, in some embodiments, LEDs (light-emitting diodes) are controlled (singly and/or in multiples) to dynamically produce a desired range of color spectra, with the ability to adjust both intensity and timing (in addition to color). For example, a semiconductor chip containing LEDs for emitting different wavelengths of light, and circuitry for controlling the light emission, can be easily integrated into virtually any lighting device or any equipment that features or that can feature internal lighting. Depending on the type of lighting devices in use (e.g., indirect wall sconces, ceiling lights, table/floor lamps), discrete color control within a relatively narrow range of spectral distribution can be afforded to an area of any size to achieve the desired outcome. An LED chip or other similar lighting component could also be integrated into specialized equipment and supporting hardware, such as an MRI machines.

The foregoing embodiments have been presented for purposes of illustration and description only. They are not intended to be exhaustive or to limit this disclosure to the forms disclosed. Accordingly, many modifications and variations will be apparent to practitioners skilled in the art. The scope is defined by the appended claims, not the preceding disclosure.

1. An apparatus comprising:

2. one or more light-emitting devices; and
a controller for controlling total light emitted by the light-emitting devices to mitigate stress experienced by one or more persons that observe the total light.

2. The apparatus of claim 1, wherein the one or more light-2 emitting devices comprise multiple light-emitting diodes (LEDs).

3. The apparatus of claim 1, wherein the light-emitting devices are selectively operable to cause the total light to comprise a single region of the visible light spectrum.

4. The apparatus of claim 3, wherein the single region of the visible light spectrum comprises a wavelength region of 570-680 nm.

5. The apparatus of claim 1, wherein the light-emitting devices are selectively operable to cause the total light to comprise a mixture of multiple regions of the visible light spectrum while omitting one or more other regions of the visible light spectrum.

6. The apparatus of claim 5, wherein:
the multiple regions comprise a wavelength region of 570-680 nm and one or more of (a) a wavelength region of 510-540 nm and (b) a wavelength region greater than 680 nm.

7. The apparatus of claim 6, wherein the one or more other regions comprise a wavelength region below 510 nm.

8. The apparatus of claim 1, wherein the light-emitting devices comprise one or more area lighting fixtures providing indirect lighting for the one or more persons.

9. The apparatus of claim 1, wherein the light-emitting devices comprise one or more area lamps.

10. The apparatus of claim 1, wherein the apparatus is a vehicle.

11. The apparatus of claim 1, wherein the apparatus comprises medical equipment.

12. The apparatus of claim 11, wherein the medical equipment comprises at least one of:
an MRI (Magnetic Resonance Imaging) machine;
a CT (Computerized Tomography) scanner;
an X-ray machine;
transfusion equipment; and
a medical display device.

13. A method comprising:
activating one or more light sources capable of emitting light in selected wavelength regions of the visible light spectrum; and
controlling the total emission of the one or more light sources to reduce stress in a human.

14. The method of claim 13, wherein said controlling comprises controlling the light sources so that a first wavelength region of 570-680 nm comprises at least 80% of the total emission.

15. The method of claim 14, wherein said controlling further comprises controlling the light sources so that:
a wavelength region of 510-540 nm comprises no more than 5% of the total emission.

16. The method of claim 14, wherein said controlling further comprises controlling the light sources so that:
a wavelength region above 680 nm comprises no more than 10% of the total emission.

17. The method of claim 14, wherein said controlling further comprises controlling the light sources so that:
the total emission is devoid of light in a wavelength region below 510 nm.

18. The method of claim 13, further comprising:
determining a stress level of the human subject; and
adjusting the total emission according to the measured stress level.

19. The method of claim 18, wherein adjusting the total emission comprises increasing the proportion of the first wavelength region.

20. The method of claim 13, wherein said controlling comprises increasing or decreasing an intensity of one or more wavelength regions of the total emission.

21. A vehicle comprising:
seating for one or more persons;
one or more light-emitting devices; and
a controller for controlling a composition of the light emitted by the one or more light-emitting devise to reduce stress levels of the one or more persons.

22. The vehicle of claim 21, wherein said controller is operable to cause the light emitted by the one or more light-emitting devices to comprise one or more of:

4. green flux in a wavelength region of 510-540 nm;
amber flux in a wavelength region of 570-680 nm; and

6. red flux in a wavelength region greater than 680 nm.

23. The vehicle of claim 22, wherein:

2. said amber flux comprises no less than 80% of the light emitted by the one or more light-emitting devices;

4. said green flux comprises no more than 5% of the light emitted by the one or more light-emitting devices; and

6. said red flux comprises no more than 10% of the light emitted by the one or more light-emitting devices.

24. The vehicle of claim 22, wherein the light emitted by the one or more light-emitting devices is devoid of light flux below 510 nm.

25. The vehicle of claim 21, wherein a composition of the light emitted by the one or more light-emitting devices is selectable by the one or more persons.

26. The vehicle of claim 21, wherein a composition of the light emitted by the one or more light-emitting devices is automatically adjusted by the controller based on a determination of a stress level of the one or more persons.
